(19) United States
(12) Patent Application Publication
(10) Pub. No.: US 2018/0055551 A1
(43) Pub. Date: Mar. 1, 2018

(54) COMPRESSION BONE SCREW

(71) Applicant: Matthew Adam Yalizis, Sans Souci (AU)

(72) Inventor: Matthew Adam Yalizis, Sans Souci (AU)

(21) Appl. No.: 15/481,911

(22) Filed: Apr. 7, 2017

Related U.S. Application Data
(62) Division of application No. 13/808,752, filed on Aug. 21, 2013, now abandoned.

(30) Foreign Application Priority Data
Jul. 7, 2010 (AU) 2010903020
Mar. 7, 2011 (AU) 2011900800

(51) Int. Cl. A61B 17/86

(52) U.S. Cl. CPC A61B 17/8625; A61B 17/8875

(57) ABSTRACT

A compression bone screw and a method of use thereof including a shank having longitudinally opposing first and second shank portions. The first shank portion is externally threaded with a first shank thread. The compression bone screw also including a first element threadingly mounted on the first shank portion by way of a first element internal thread threadingly engaging the first shank thread. The first element is externally threaded with a first element external thread and a second element is integrally formed with and by an enlargement of the second shank portion. The second element is externally threaded with a second element external thread, and the second element external thread and the first element external thread are like-handed.

### TECHNICAL FIELD

The present invention relates to the field of bone fixation in orthopaedic surgery and in particular relates to a compression bone screw.

### BACKGROUND OF THE INVENTION

Compression bone screws are used in orthopaedic surgery for bone fixation, and in particular to fix two fragments of a fractured bone and for fixation of osteotomies and arthrodeses. Healing of fractured bones is greatly improved with the application of compression between the bone fragments.

Various forms of compression bone screw have previously been proposed seeking to provide such compression between bone fragments. One form of known compression bone screw, known as the Herbert screw, utilises external leading and trailing threads on the screw for embedment within distal and proximal bone fragments. The leading and trailing external threads are like-handed, but are of unequal pitch, with a greater pitch on the leading thread. This advances the screw through the distal bone fragment at a greater rate than through the proximal bone fragment, thereby drawing the distal bone fragment towards the proximal bone fragment, providing a degree of compression between the bone fragments. The amount of compression provided, however, cannot be readily controlled and is entirely dependent upon the thread pitch differentiation and the depth to which the screw must be embedded to provide adequate purchase on the distal and proximal bone fragments.

Another previously proposed form of compression bone screw utilises separate externally threaded leading and trailing elements for embedment within the distal and proximal bone fragments. The leading and trailing elements are rotatable independently of each other and configured such that they may be releasably fixed relative to each other such that they rotate in unison. Such screws are implanted by first fixing the leading and trailing elements and threadingly driving the screw into the proximal and distal bone fragments until the leading element is firmly embedded within the distal bone fragment and the trailing element is firmly embedded within the proximal bone fragment. The leading element is then unlocked from the trailing element and the leading element rotationally driven independent of the trailing element, tending to continue driving the leading element through the distal bone fragment.

Given that the trailing element remains fixed in the proximal fragment, the rotation of the leading element results in the distal bone fragment being drawn back towards the proximal bone fragment, providing compression between the two bone fragments. With this arrangement, however, the second action, tending to continue driving the leading element through the distal fragment, may result in the leading element projecting from the distal surface of the distal bone fragment and reducing the degree of embedment of the leading element within the distal bone fragment. The typical coarse pitch thread, designed to cut into the bone material, may also result in failure in poor quality bone material when attempting to generate significant compression loads by driving the coarse steel thread further through the bone. The coarse thread also limits the degree of control of the amount of compression applied.

### OBJECT OF THE INVENTION

It is an object of the present invention to substantially overcome or at least ameliorate at least one of the above disadvantages.

### SUMMARY OF THE INVENTION

In a first aspect, the present invention provides a compression bone screw comprising:

a shank having longitudinally opposing first and second shank portions, said first shank portion being externally threaded with a first shank thread;
a first element threadingly mounted on said first shank portion by way of a first element internal thread threadingly engaging said first shank thread, said first element being externally threaded with a first element external thread;
a second element mounted on, or integrally formed with, said second shank portion, said second element being externally threaded with a second element external thread, said second element external thread and said first element external thread being like-handed; and
said first shank portion and said first element being configured to operate in a first mode of operation in which said shank and said first element are rotationally driven in unison and a second mode of operation in which said shank is rotationally driven independent of said first element, such that said first element moves along said first shank portion towards said second element;
wherein a pitch of said first element external thread is substantially equal to a pitch of said second element external thread.

In a preferred form, said first element external thread and said second element external thread are each self-tapping threads.

In a preferred form, said second element external thread extends along substantially the entire length of said second element.

Typically, said pitch of said first and second element external threads is coarser than the pitch of said first shank thread.

In one or more preferred embodiments, said first shank portion is a trailing shank portion, said second shank portion is a leading shank portion, said first element is a trailing element and said second element is a leading element.

In a first embodiment, said second element is fixedly mounted on, or integrally formed with, said second shank portion.

In one or more embodiments, said first element external thread is opposite-handed to said first shank thread.

In one form, an end face of said first shank portion is provided with a primary drive formation and an end face of said first element is provided with a secondary drive formation, said primary and secondary drive formations being engageable with a drive tool in said first mode of operation for rotationally driving said shank and said first element in unison, said primary drive formation being engageable with the drive tool in said second mode of operation for rotationally driving said shank independently of said first element.

Typically, said primary drive formation is engageable with a primary drive head of the same drive tool in said first and second modes of operation, said secondary drive formation being engageable with a secondary drive head of the drive tool in said first mode of operation and disengageable from the secondary drive head in said second mode of operation. Alternatively, the primary drive head may be rotatable independently of the secondary drive head in the second mode of operation.

In one form, said secondary drive formation is in the form of a plurality of slots formed in an end face of said first element.

In one embodiment, in which said first element is a trailing element, said first element external thread has a first element external thread outer diameter that tapers towards a leading end of said first element.

In at least one embodiment, in which said first element is a trailing element, at least a leading region of said first shank thread tapers towards a trailing end of said shank and at least a leading region of said first element is configured to radially expand upon engagement with said leading region of said first shank thread. Typically, at least a leading region of said first element is provided with one or more longitudinally extending slits to enable radial expansion of said leading region of said first element.

Preferably, said compression bone screw further comprises a locking mechanism Configured to lock said first element relative to said first shank portion upon installation of said compression bone screw.

In one form, said locking mechanism comprises a deformable detent secured to one of said first element and said first shank portion such that, upon installation of said compression bone screw, the other of said first element and said first shank portion engages and deforms said deformable detent to lock said first element to said first shank portion.

In one form, the locking mechanism comprises a locking device configured to engage said end face of said first shank portion and said end face of said first element to lock said first element relative to said first shank portion upon installation of said compression bone screw. Typically, said locking device engages said primary drive formation and said secondary drive formation.

In one form, said first shank portion and said first element are each configured to releasably engage a locking member to fix said first shank portion relative to said first element for said first mode of operation.

In such a form, typically said first shank portion is provided with a longitudinally extending shank groove and said first element is provided with a first element groove, said shank grove and said first element groove co-operating to receive said locking member in use, The locking member will typically be in the form of an elongate pin or screw.

In one or more embodiments:

said second shank portion is externally threaded with a second shank thread, said second shank thread being opposite-handed to said first shank thread, said second element being threadingly mounted on said second shank portion by way of a second element internal thread threadingly engaging said second shank thread; and
said second shank portion and said second element are configured to be rotationally driven in unison in said first mode of operation and such that said shank is rotationally driven independent of said second element in said second mode of operation such that said second element moves along said second shank portion towards said first element.

In one form, said shank, said first element and said second element are each configured to releasably engage a locking member to fix said shank relative to said first element and said second element for said first mode of operation.

In a preferred form, said locking member forms said locking mechanism.

In a preferred form, said shank is provided with a shank groove longitudinally extending along said first and second shank portions, said first element is provided with a first element groove, and said second element is provided with a second element groove, said shank groove, said first element groove and said second element groove co-operating to receive said locking member in use, said locking member being in the form of an elongate pin.

In at least one embodiment, first and second primary detents are provided at the end of said first and second shank portions respectively for engaging said first element and said second element respectively during said first mode of operation, thereby enabling said shank to be rotationally driven in unison with said first element and said second element in said first mode of operation, said second shank thread being opposite handed to said second element external thread.

In one embodiment, first and second secondary detents are provided between said first and second shank portions for engaging said first element and said second element respectively upon completion of said second mode of operation, thereby limiting movement of said first element towards said second element. In a preferred form, said first and second secondary detents are configured such that, upon engagement with said first and second elements respectively, said shank does not longitudinally extend beyond said first element or said second element.

In a second aspect, the present invention provides a compression bone screw comprising:

a shank having longitudinally opposing first and second shank portions, said first shank portion being externally threaded with a first shank thread, said second shank portion being externally threaded with a second shank thread, said second shank thread being opposite-handed to said first shank thread;
a first element threadingly mounted on said first shank portion by way of a first element internal thread threadingly engaging said first shank thread, said first element being externally threaded with a first element external thread; and
a second element threadingly mounted on said second shank portion by way of a second element internal thread threadingly engaging said second shank thread, said second element being externally threaded with a second element external thread, said second element external thread and said first element external thread being like-handed; said shank, said first element and said second element being configured to operate in a first mode of operation in which said shank, said first element and said second element are rotationally driven in unison and a second mode of operation in which said shank is rotationally driven independent of said first element and said second element, such that said first element moves along said first shank portion towards said second element and said second element moves along said second shank portion towards said first element.

In one form, said first shank portion is a trailing shank portion, said second shank portion is a leading shank portion, said first element is a trailing element and said second element is a leading element.

In one specific embodiment, said first element external thread is configured to engage a mating thread of an aperture extending through a locking plate and said second element external thread is a self-tapping thread for engaging bone.

In one form, said shank, said first element and said second element are each configured to releasably engage a locking member to fix said shank relative to said first element and said second element for said first mode of operation.

In such a form, typically said shank is provided with a shank groove longitudinally extending along said first and second shank portions, said first element is provided with a first element groove, and said second element is provided with a second element groove, said shank groove, said first element groove and said second element groove co-operating to receive the locking member in use. The locking member will typically be in the form of an elongate pin or screw.

In one form first and second primary detents are provided at the end of said first and second shank portions respectively for engaging said first element and said second element respectively during said first mode of operation, thereby enabling said shank to be rotationally driven in unison with said first element and said second element in said first mode of operation, said second shank thread being opposite handed to said second element external thread.

In one form, first and second secondary detents are provided between said first and second shank portions for engaging said first element and said second element respectively upon completion of said second mode of operation, thereby limiting movement of said first element towards said second element.

In a third aspect, the present invention provides a method of fixing a proximal bone fragment to a distal bone fragment, said method comprising the steps of:

a) providing a compression bone screw comprising:
(i) a shank having longitudinally opposing first and second shank portions, said first shank portion being externally threaded with a first shank thread;
(ii) a first element threadingly mounted on said first shank portion by way of a first element internal thread threadingly engaging said first shank thread, said first element being externally threaded with a first element external thread; and
(iii) a second element mounted on, or integrally formed with, said second shank portion, said second element being externally threaded with a second element external thread, said second element external thread and said first element external thread being like-handed;
b) drilling a hole through said proximal bone fragment into said distal bone fragment;
c) rotationally driving said screw into said hole with said second element leading, rotationally driving said first element, said second element and said shank in unison until said second element is embedded within said distal bone fragment and said first element is embedded in said proximal bone fragment;
d) rotationally driving said shank independently of said first element in a direction tending to draw said first and second elements together.

In one form, said second element is fixedly mounted on, or integrally formed with, said second shank portion, and in step (c) said shank is rotationally driven in unison with said second element.

In an alternate form, said second shank portion is externally threaded with a second shank thread, said second shank thread being opposite-handed to said first shank thread, said second element being threadingly mounted on said second shank portion by way of a second element internal thread threadingly engaging said second shank thread and, in step (c) said shank is rotationally driven independently of said second element.

Also disclosed is a method of fixing a proximal bone fragment to a distal bone fragment, said method comprising the steps of:

a) drilling a hole through said proximal bone fragment into said distal bone fragment;
b) securing a locking plate to an adjacent stable bone portion, aligning a threaded aperture extending through said locking plate with said hole;
c) providing a compression bone screw comprising:
(i) a shank having longitudinally opposing first and second shank portions, said first shank portion being externally threaded with a first shank thread, said second shank portion being externally threaded with a second shank thread, said second shank thread being opposite-handed to said first shank thread;
(ii) a first element threadingly mounted on said first shank portion by way of a first element internal thread threadingly engaging said first shank thread, said first element being externally threaded with a first element external thread matching the thread of said threaded aperture; and
(iii) a second element threadingly mounted on said second shank portion by way of a second element internal thread threadingly engaging said second shank thread, said second element being externally threaded with a second element external thread, said second element external thread and said first element external thread being like-handed;
d) rotationally driving said screw through said threaded aperture into said hole with said second element leading, rotationally driving said first element, said second element and said shank in unison until said second element is embedded within said distal bone fragment and said first element is embedded within said threaded aperture;
e) rotationally driving said shank independently of said first element in a direction tending to draw said first and second elements together.

### BRIEF DESCRIPTION OF THE DRAWINGS

Preferred embodiments of the present invention will now be described, by way of example only, with reference to the accompanying drawings wherein:

FIG. 1 is a front elevation view of a compression bone screw according to a first embodiment;

FIG. 2 is an end elevation view of the compression bone screw of FIG. 1;

FIG. 3 is an opposing end elevation view of the compression bone screw of FIG. 1;

FIG. 4 is an isometric view of the compression bone screw of FIG. 1;

FIG. 5 is a schematic cross-sectional view of the compression bone screw of FIG. 1;

FIG. 6 is a partially cross-sectioned view of a drive tool for use with the compression bone screw of FIG. 1;

FIG. 7 is a schematic view of a fractured bone with the compression bone screw of FIG. 1 partly implanted;

FIG. 8 is a schematic view of the fractured bone of FIG. 7 with the compression screw of FIG. 1 fully implanted;

FIG. 9 is a side elevation of a compression bone screw according to a third embodiment;

FIG. 10 is an isometric view of the compression bone screw of FIG. 9;

FIG. 11 is a further isometric view of the compression bone screw of FIG. 9;

FIG. 12 is a schematic cross-sectional view of the compression bone screw of FIG. 9;

FIG. 13 is a schematic cross-sectional view of the compression bone screw of FIG. 9 in an implanted configuration;

FIG. 14 is a schematic view of an ankle arthrodesis with the compression bone screw of FIG. 9 partly implanted;

FIG. 15 is a schematic view of the ankle arthrodesis of FIG. 14 with the compression bone screw of FIG. 9 fully implanted;

FIG. 16 is a schematic perspective view of a trailing end portion of the compression bone screw of FIG. 9;

FIG. 17 is a perspective view of a locking device for use with the compression bone screw of FIG. 9;

### DETAILED DESCRIPTION OF THE PREFERRED EMBODIMENTS

A compression bone screw 100 according to a first embodiment is depicted in FIGS. 1 to 5. The compression bone screw 100 comprises a shank 110, a first element 120 and a second element 130.

The shank 110 has longitudinally opposing first and second shank portions 111, 112. The first shank portion 111 is externally threaded with a first shank thread 113 that is here a left-handed thread. In the arrangement depicted, the shank 110 has an overall diameter of approximately 6 mm and the first shank thread 113 has a relatively fine pitch of approximately 1 mm.

The first element 120 has a first element aperture 121 longitudinally extending therethrough and a first element internal thread 122 formed on the wall of the first element aperture 121, The first element 120 is threadingly mounted on the first shank portion 111 by way of the first element internal thread 122 threadingly engaging the first shank thread 113. Accordingly, the first element internal thread 122 and first shank thread 113 are matching and are here in the form of machine threads. The first element 120 is also externally threaded with a first element external thread 123. The first element external thread 123 will typically extend along substantially the entire length of the first element 120. In the arrangement depicted, the first element external thread 123 is opposite-handed to the first shank thread 113, being a right-handed thread. The first element external thread 123 here has a relatively course pitch of approximately 2 mm, (compared to a relatively finer 1 mm pitch of the first shank thread 113), a thread depth of approximately 1.5 mm, and is self-tapping for screwing into bone material. In the arrangement depicted, the first element 120 typically has an overall diameter of approximately 11 mm and a length of the order of 15 mm.

In the first embodiment, the second element 130 is fixedly mounted on the second shank portion 112. The second element 130 could be fixed, typically by welding, to the end of the second shank portion 112 or alternatively the second shank portion 112 may project into a recess formed in the end of the second element 130 and be fixed thereto. It is also envisaged that the second element 130 might be integrally formed with the shank second portion 112, typically by machining, as a monoblock construction. The second element 130 is externally threaded with a second element external thread 133 that is like-handed to the first element external thread 123 (right-handed in the arrangement depicted) and an at least substantially identical pitch. The second element external thread 133 is again a self-tapping thread and will typically extend along substantially the entire length of the second element 130. The second element 130 has an equivalent length to that of the first element 120, being a length of approximately 15 mm and has a smaller overall diameter of approximately 10 mm, so that the first element external thread 123 is able to get a better purchase on the bone material after the second element external thread 133 has already passed therethrough. It is also envisaged, however, that the first and second elements 120, 130 may have the same overall diameter. It is further envisaged that the first and second elements 120, 130 may have differing lengths to suit applicability to different bones and clinical scenarios.

To protect against loosening of the compression bone screw 100 after implantation, the compression bone screw 100 here further comprises a locking mechanism configured to lock the first element 120 relative to the first shank portion 111 upon installation of the screw 100. In the first embodiment, the locking mechanism comprises a deformable detent 160 secured to the interior of the first element 120. As depicted in FIG. 5, the detent 160 is here positioned adjacent the trailing first element end face 124 such that, as will be described below, upon installation of the compression bone screw 100 the first shank portion 111 engages the detent 160 and deforms the detent 160 so as to lock the first element 120 to the first shank portion 111. In the particular arrangement depicted, the deformable detent 160 is in the specific form of a collar insert which may be formed of nylon or another deformable material, in a similar manner to the nylon collar insert of a standard lock nut. Rather than being mounted internally on the first element 120, it is also envisaged that an equivalent deformable detent 160′, again as depicted in FIG. 5, could be mounted on the first shank portion 111, towards the second shank portion 112, so as to engage the first element 120 in a similar manner.

In the arrangement depicted, the second element 130 is the leading element of the compression bone screw 100 intended to lead when screwed into a fractured bone and, accordingly, is provided with a tapered self-tapping point 134 for drilling into bone material. The first element 120 is thus the trailing element, the first shank portion 111 is the trailing shank portion and the second shank portion 112 is the leading shank portion.

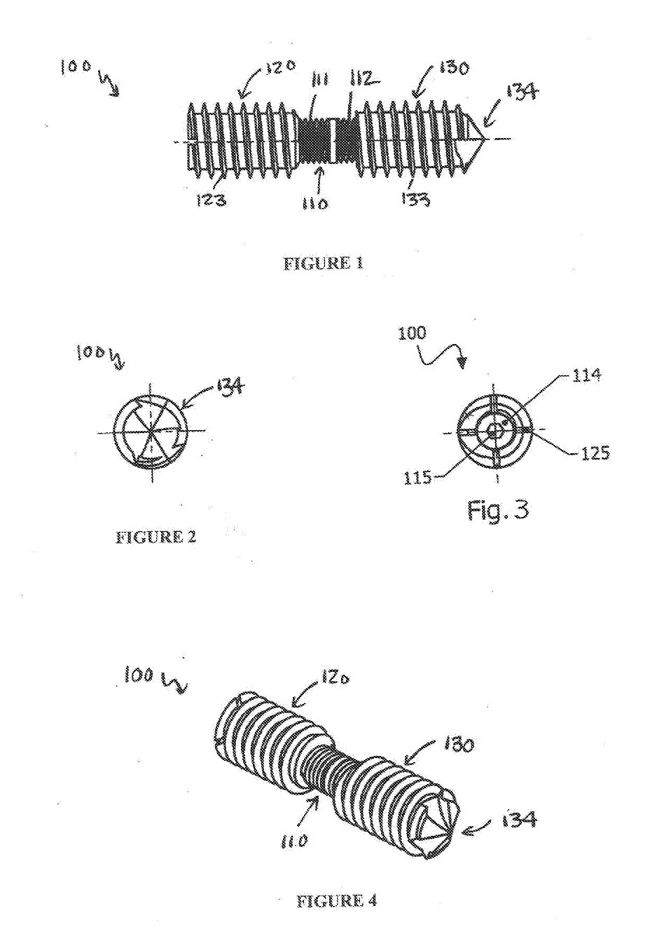

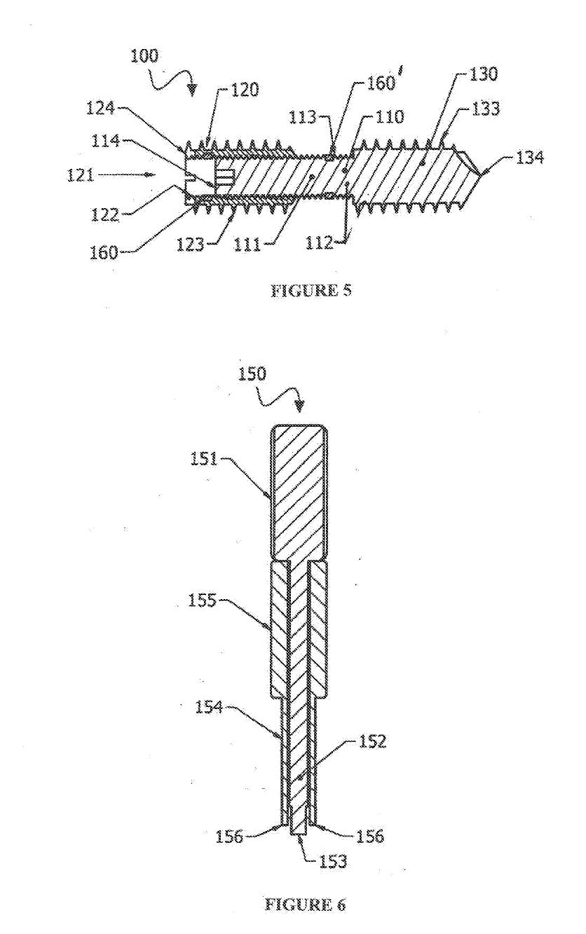

With the second element 130 being fixedly mounted on the second shank portion 112, the second element 130 rotates in unison with the shank 110 during use. The first shank portion 111 and first element 120 are configured to operate in a first mode of operation, as will be discussed below, in which the shank 110 and first element 120 are rotationally driven in unison and in a second mode of operation, as will again be discussed further below, in which the shank 110 is rotationally driven independent of the first element 120. In the first embodiment, these two modes of operation are provided for by way of primary and secondary drive formations provided on the first element 120 and the first shank portion 111 and an associated driving tool 150 depicted in FIG. 6. The first shank portion 111 has a first shank end face 114 provided with a primary drive formation, here in the form of a hexagonal drive socket 115 (see FIG. 3). The primary drive formation might, however, take any form of drive formation including a slot, Phillips head, star drive or other polygon drive form. The first element 120 has an annular trailing first element end face 124 that is provided with a secondary drive formation, here in the form of a plurality of slots 125, here configured as four equally spaced slots 125, formed in the first element end face 124. Again, the secondary drive formation may take any suitable drive formation form.

The drive tool 150 is formed with a handle 151 and elongate shaft 152 on the end of which is formed a hexagonal primary drive head 153 that is configured to engage the hexagonal drive socket 115 of the first shank portion 111. The drive tool 150 is further provided with a sleeve 154 that is mounted on the shaft 152, here so as to be longitudinally displaceable along the shaft 152 by way of finger grips 155 formed on the trailing end of the sleeve 154 for activation by a user gripping the handle 151. At the leading end of the sleeve 154 is provided a secondary drive head comprising four lugs 156 configured to engage the slots 125 provided in the first element end face 124. With the hexagonal primary drive head 153 engaging the hexagonal drive socket 115, the sleeve 154 may be advanced such that the lugs 156 engage the slots 125, for the first mode of operation to rotationally drive the first element 120 and shank 110 in unison. The sleeve 154 may be retracted, with the hexagonal primary drive head 153 still engaging the hexagonal drive socket 115, such that the lugs 156 disengage the slots 125, thereby allowing the shank 110 to be rotationally driven independently of the first element 120 in the second mode of operation. Alternatively, the sleeve 154 could be non-retractable and held stationary in engagement with the lugs 156 whilst the primary drive head 153 drives the shank 110 in the second mode of operation. Rather than providing the secondary drive head lugs 156 on the end of a retractable sleeve 154, it is also envisaged that two separate drive tools might be utilised, one having a drive head for engaging the primary drive formation only for the first mode of operation and the other having either a single drive head configured to engage both the primary and secondary drive formations, or separate drive heads for engaging each of the primary and secondary drive formations for the second mode of operation.

An alternate configuration is envisaged where the first element 120 forms the leading element and the second element 130 forms the trailing element. In such a configuration however, the first element 120, being the element threadingly mounted on the shank 110, would not be as accessible for access by a drive tool for driving the first element 120 in unison with the first shank portion 111. As an alternative, the first element 120 could be releasably fixed to the first shank portion 111 for the first mode of operation in a similar manner to that described below in relation to the second embodiment.

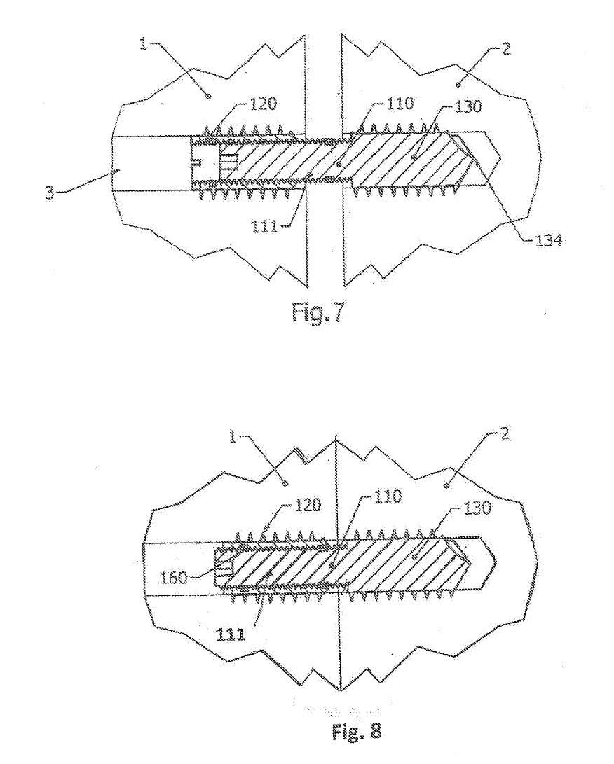

The fixing of proximal and distal bone fragments 1, 2 of a fractured bone using the compression bone screw 100 of the first embodiment will now be described with reference to FIGS. 7 and 8.

With the proximal and distal bone fragments 1, 2 appropriately positioned, a guide hole 3 with a diameter of approximately 8 mm for the arrangement depicted is first drilled through both the proximal and distal bone fragments 1, 2. The guide hole 3 may extend through the thickness of the distal fragment 2, or may be a blind hole as depicted. The guide hole 3 may then be pre-tapped, particularly if the bone material is particularly hard or if the first and second element external threads 123, 133 are not of a self-tapping configuration. The tapered point 134 of the second element 130 is then inserted into the guide hole 3 formed in the proximal bone fragment 1. The drive tool 150 is engaged with the compression bone screw 100, engaging the hexagonal primary drive head 153 with the hexagonal drive socket 115 formed in the end face 114 of the first shank portion 111 and the sleeve 154 of the drive tool 150 is advanced such that the lugs 156 engage the slots 125 formed in the end face 124 of the first element 120. The drive tool 150 is then rotationally driven by hand in a clockwise direction in the first mode of operation, thereby rotationally driving the first element 120 in unison with the shank 110 (and second element 130), advancing the entire compression bone screw 100 through the guide hole 3. With the pitch of the first element external thread 123 and second element external thread 133 being at least substantially identical, the first and second elements 120, 130, rotating in unison, will advance through the proximal and distal bone fragments 1, 2 respectively at at least a substantially equal rate so that the first mode of operation will not tend to either draw the proximal and distal bone fragments 1, 2 towards each either or drive them apart. The compression bone screw 100 is rotationally driven until the second element 130 is firmly embedded within the distal bone fragment 2 and the first element 120 is firmly embedded within the proximal bone fragment 1.

At this point, the proximal and distal bone fragments 1, 2 will be firmly fixed in relation to each other, but without any compression being applied at the interface between the bone fragments 1, 2. The sleeve 154 is then retracted so as to disengage the lugs 156 from the slots 125 (or kept engaged with the lugs 156 but held stationary). The drive tool 150 is then further rotationally driven in the clockwise direction in the second mode of operation (or at least the shaft 152 rotationally driven). This results in rotation of the shank 110 together with the second element 130, independently of the first element 120 which remains stationary within its embedded position within the proximal bone fragment 1. The rotation of the second element 130 tends to advance the second element 130 deeper into the distal bone fragment 2 by virtue of the right-handed thread of the second element external thread 133, thereby effectively drawing the distal bone fragment 2 back toward the proximal bone fragment 1. At the same time, rotation of the shank 110 tends to retract the first shank portion 111 relative to the first element 120 by virtue of the opposite left-handed thread of the first shank thread 113. The first element 120 thus moves forward along the first shank portion 111 towards the second element 130. This draws the proximal bone fragment 1 toward the distal bone fragment 2, generating the desired compression between the bone fragments 1, 2, without displacement of the first element 120 within the proximal bone fragment 1. Having the first shank thread 113 and second element external thread 133 opposite-handed ensures that the relative motion of the proximal and distal bone fragments 1, 2 towards each other in a single revolution of the shank 110 is equal to the sum of the pitches of the two threads. Accordingly, a desired level of compression can be achieved through significantly less revolutions than is required with compression bone screws that rely on unequal pitched threads to achieve compression, Both the first and second element external threads 123, 133 being made with substantially the same coarse pitch, provide improved fixation in cancellous bone.

When the first shank portion 111 is drawn sufficiently back into the first element 120 during the second mode of operation, the first shank portion 111 engages the deformable detent 160 to lock the first element 120 onto the first shank portion 111, thereby inhibiting loosening of the compression bone screw 100 after implementation which might otherwise result from micro-movement of the proximal and distal bone fragments 1, 2 during the healing process.

The implantation of the compression bone screw 100 may be conducted with the assistance of a guide wire extended through the guide hole 3. Where a guide wire is to be utilised, a bore hole (not depicted) will extend through the centre of the shank 110 and the second element 130 for receipt of the guide wire 3. Similarly, a bore hole will extend through the centre of the drive tool 150. The drive tool 150 and compression bone screw 100 will thus be mounted on the guide wire throughout the implantation process.

It is further envisaged that, in both embodiments described above, the first and second element external threads may be left-handed rather than right-handed, counter-clockwise driving of the drive tool would again be required and, in the first embodiment, the first shank thread would be right-handed.

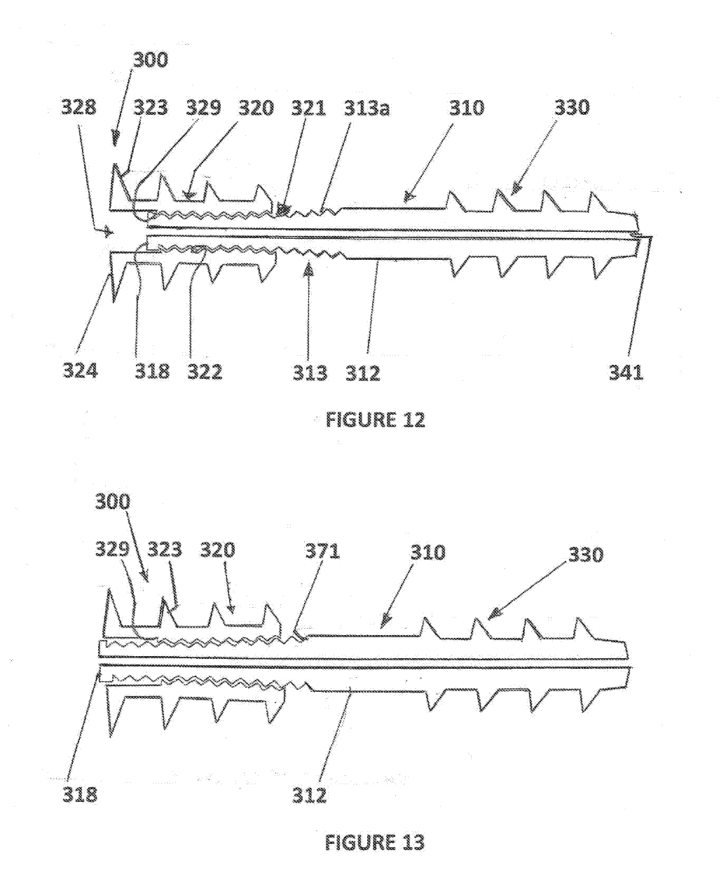

A compression bone screw 300 according to a third embodiment is depicted in FIGS. 12 to 16. The compression bone screw 300 is a modified version of the compression bone screw 100 of the first embodiment and shares the same basic configuration. Those features of the compression bone screw 300 of the third embodiment that are identical, or equivalent, to features of the compression bone screw 100 of the first embodiment are again provided with like reference numerals, here increased by 200. The compression bone screw 300 comprises a shank 310, a first element 320 and a second element 330. The shank 310, first element 320 and second element 330 may be formed of the same materials as per the first and second embodiments. In the arrangement depicted, the first element 320 is the trailing element, the first shank portion 311 is the trailing shank portion and the second shank portion 312 is the leading shank portion.

The shank 310 has longitudinally opposing first and second shank portions 311, 312. The first shank portion 311 is externally threaded with a first shank thread 313 that is again a left-handed thread. A leading region 313a of the first shank thread 313 is tapered, with the first shank thread 313 tapering in this region towards the first shank end face 314 at a taper angle that will typically be about 2 to 3 degrees. The taper angle may, however be larger, with angles up to at least 30 degrees being envisaged. Here the first shank thread 313 has an overall diameter of approximately 4.0 mm at its leading end adjacent the second shank portion 312, and an overall diameter of approximately 3.4 mm along the length of the trailing region of the first shank thread 313. As with the first embodiment, the first shank thread 313 here has a pitch of approximately 1 mm. The second shank portion 312 is here of a cylindrical non-threaded form. A central bore hole 341 sized to receive a guide wire is provided along the length of the shank 310, as would typically be the case with the compression bone screw 100 and 200 of the first and second embodiments (although not depicted). A first primary detent 318, in the form of an enlarged head, is formed at the trailing first shank end face 314.

The first element 320 has a first element aperture 321 longitudinally extending therethrough and a first element internal thread 322 formed on the wall of the first element aperture 321. The first element 320 is threadingly mounted on the first shank portion 311 by way of the first element internal thread 322 threadingly engaging the first shank thread 313, as per the first embodiment. The first element 320 is externally threaded with a first element external thread 323 which again extends along substantially the entire length of the first element 320. In this embodiment, the first element external thread 323 is opposite-handed to the first shank thread 313, being a right-handed thread. The first element external thread 323 here has a relatively coarse pitch of approximately 2 mm and an overall diameter that tapers towards a leading end of the first element 320. To achieve this tapering of the first element thread overall diameter, the thread depth decreases towards the leading end of the first element 320, here decreasing from approximately 2.8 mm to approximately 1.5 min. The first element thread 323 is also self-tapping for screwing into bone material. The first element 320 typically has an overall diameter of approximately 10.5 mm its trailing end and a length of the order of 15 mm. The trailing first element end face 314 is provided with a first element recess 328 for receipt of the first primary detent 318. The base of the first element recess 328 forms an annular first element shoulder 329 which engages the first primary detent 318 at one end of the extent of travel of the first element 320 along the first shank portion 311, to retain the first element 320 on the first shank portion 311. Travel of the first element 320 along the first shank portion 311 in the opposing direction is limited by engagement of the leading end of the first element 320 with a first secondary detent 371 defined at the junction between the threaded first shank portion 311 and the non-threaded second shank portion 312. The first secondary detent is effectively defined by the runout of the first shank thread 313. The first element recess 328 has a length such that the first primary detent 318 does not project beyond the first element recess 328 at any point along the extent of travel of the first element 320, ensuring that there is no overhang of the shank 310 from the first element 320 upon implantation.

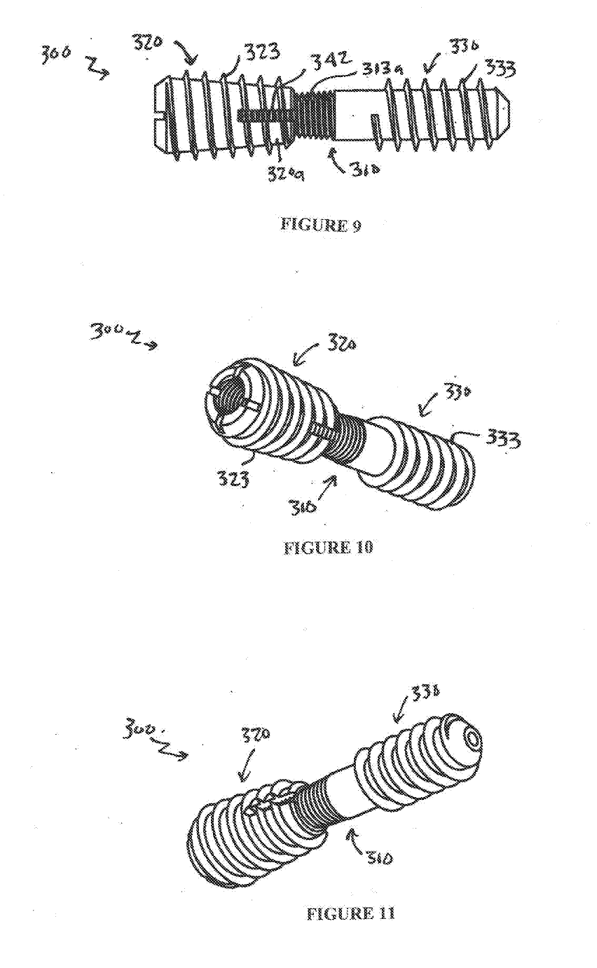

Referring specifically to FIG. 9, the first element 320 is provided with one or more longitudinally extending slits 342 extending from the leading end of the first element 320, along about 30 to 50 percent of the length of the first element, to enable radial expansion of the leading region 320a of the first element 320 as it is advanced along the tapered leading region 313a of the first shank thread 313 during the second mode of operation of the compression bone screw 300 as will be discussed below.

The second element 330 is fixed in relation to the shank 310, here specifically being integrally formed with the shank 310 such that the second element 330 and shank rotate in unison during use. The second element 330 is of the same general configuration as the second element 130 of the compression bone screw 100 of the first embodiment, being externally threaded with a second element external thread 333 that is like-handed to the first element external thread 323 (right-handed in the arrangement depicted) and which extends along substantially the entire length of the second element 330. The second element external thread 333 again typically has a substantially identical pitch to that of the first element external thread 323 and has a diameter slightly less than that of the leading end of the first element 320, so that the first element external thread 323 is able to get a better purchase on the bone material after the second element external thread 333 has already passed therethrough.

Again as with the first embodiment, the first shank portion 311 and first element 320 are configured to operate in a first mode of operation in which the shank 310 and first element 320 are rotationally driven in unison and in a second mode of operation in which the shank 310 is rotationally driven independent of the first element 320. Again similar to the first embodiment, these two modes of operation are provided for by way of primary and secondary drive formations formed on the first element 320 and the first shank portion 311 respectively and an associated driving tool equivalent to that of the driving tool 150 depicted in FIG. 6. The first shank end face 314 is provided with a primary drive formation, shown in FIG. 23 in the form of a slot 315, for engaging a primary drive head in the form of a blade rather than the hexagonal drive head 153 of the drive tool 150. Again, any suitable form of primary drive formation might be provided on the first shank end face 314. The annular trailing first element end face 324 is provided with a secondary drive formation in the form of a plurality of slots 325 as per the first embodiment.

The configuration of the first element 320 and first shank portion 311 of the third embodiment may also be applied to the first element 220 and first shank portion 211 of the compression bone screw 200 of the second embodiment, particularly in relation to the tapering aspects of the first element thread 323 and tapered leading region 313a of the first shank thread 313 along with the slits 342 in the first element 320.

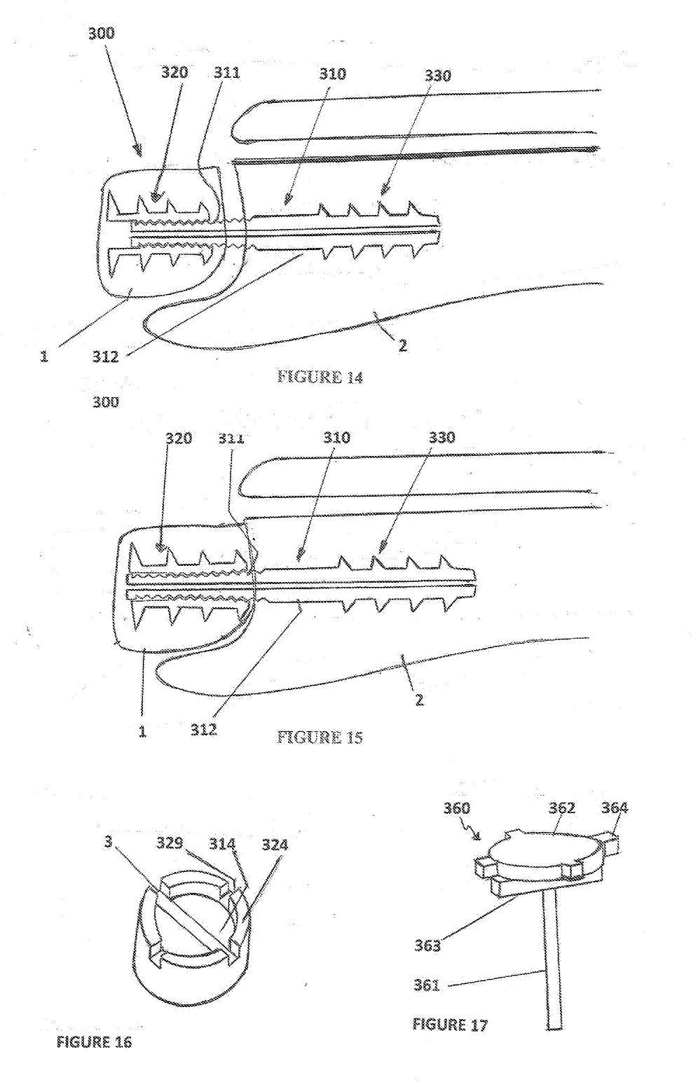

The procedure for fixing proximal and distal bone fragments 1, 2 of a fractured bone using the compression bone screw 300 of the third embodiment is generally as per the above described procedure in relation to the compression bone screw 100 of the first embodiment, and will now be described in further detail with reference to FIGS. 14 and 15.

As with the first embodiment, a guide hole 3 is first drilled through the proximal and distal bone fragments 1, 2 and the tapered self-tapping point 334 of the second element 330 inserted into the guide hole 3 formed in the proximal bone fragment 1. The drive tool 150 is engaged with the compression bone screw 300 engaging the primary slot 315 and secondary drive formation slots 325 and the drive tool 150 rotationally driven in a clockwise direction in the first mode of operation, thereby rotationally driving the first element 320 in unison with the shank 310 and second element 330, advancing the entire compression bone screw 300 through the guide hole 3. As with the procedure for the first embodiment, once the second element 330 is firmly embedded within the distal bone fragment 2 and the first element 320 is firmly embedded within the proximal bone fragment 1, the sleeve 154 of the drive tool 150 is retracted so as to disengage the lugs 156 from the secondary drive formation slots 125 and the drive tool is further rotationally driven in a clockwise direction in the second mode of operation. This results in rotation of the shank 310 together with the second element 330, independent of the first element 320 which remains stationary. Rotation of the second element 330 tends to advance the second element 330 deeper into the distal bone fragment 2, thereby effectively drawing the distal bone fragment 2 back towards the proximal bone fragment 1. At the same time, rotation of the shank 310 tends to retract the first shank portion 311 relative to the first element 320. The first element 320 thus moves forward along the first shank portion 311 towards the second element 330. This draws the proximal bone fragment 1 towards the distal bone fragment 2, generating the desired compression between the bone fragments 1, 2 without displacement of the first element 320 with any proximal bone fragments.

At the same time, as the first element is drawn along the first shank portion 311, engaging the tapered leading region 313a of the first shank thread 313, the leading region 320a of the first element 320 radially expands, by expansion of the slits 342. This radial expansion provides a compressive preload between the first element 320 and bone increasing the pullout strength of the compression bone screw 300 in weaker, particularly osteoporotic, bone, thereby increasing the strength of the overall construct. A similar preload is applied in the trailing region of the first element 320 by virtue of the taper of the first element thread 323 as the first element 320 is advanced through the proximal bone fragment 1 during the first mode of operation.

Once implantation of the compression bone screw 300 is complete, when the desired compression is achieved, the first element 320 may be locked in relation to the shank 311 by way of a locking mechanism in the form of a locking device 360 as depicted in FIG. 17. The locking device 360 has an elongate shaft 361 configured to extend into the bore hole 341 in the shank 310. The locking device also has a head 362, the leading face of which is provided with a primary locking structure, here in the form of an elongate rib 363, configured to engage the first shank end face 314, particularly the primary drive formation slot 315 of the first shank portion 311. The locking device body 362 is further provided with a secondary locking structure, here in the form of four radially projecting lugs 364, configured to engage the first element end face 324, particularly the secondary drive formation slots 325. Once in place, the locking device 360 prevents any relative rotation between the first element 320 and the first shank portion 311. An equivalent locking device may also be used with the compression bone screw 100 of the first embodiment.

Each of the components of each of the compression bone screws described above may be formed of stainless steel, titanium or any other suitable biologically inert engineering material, including bioabsorbable materials such as polylactic acid or the like. The various dimensions of each of the components of each of the compression bone screws described above may also be adjusted to suit a particular intended application.

A person of ordinary skill in the art will appreciate that various other modification and alterations of the compression screws described may be made without departing from the spirit of the invention. In particular, various other mechanisms may be utilised to provide for rotation of the shank with the first (and second) elements in the first mode of operation.

1. A compression bone screw, comprising:
a shank having longitudinally opposing first and second shank portions, said first shank portion being externally threaded with a first shank thread;
a first element threadingly mounted on said first shank portion by way of a first element internal thread threadingly engaging said first shank thread, said first element being externally threaded with a first element external thread;
a second element integrally formed with said second shank portion, said second element being externally threaded with a second element external thread, said second element external thread and said first element external thread being like-handed; and
said first shank portion and said first element being configured to operate in a first mode of operation in which said shank and said first element are rotationally driven in unison urging said first element and said second element axially in the same direction and at the same rate; and
a second mode of operation in which said shank is rotationally driven independent of said first element, such that said first element moves axially along said first shank portion towards said second element;
wherein a pitch of said first element external thread is substantially equal to a pitch of said second element external thread.

2. The screw of claim 1, wherein:
said first element external thread and said second element external thread are each self-tapping threads.

3. The screw of claim 1, wherein:
said pitch of said first and second element external threads is coarser than a pitch of said first shank thread.

4. The screw of claim 1, wherein:
said first shank portion is a trailing shank portion, said second shank portion is a leading shank portion, said first element is a trailing element and said second element of said second shank portion is a leading element.

5. The screw of claim 4, wherein:
said second element is formed as an enlarged diameter portion of the second shank portion.

6. The screw of claim 5, wherein:
said first element external thread is opposite-handed to said first shank thread.

7. The screw of claim 5, wherein:
an end face of said first shank portion is provided with a primary drive formation and an end face of said first element is provided with a secondary drive formation, said primary and secondary drive formations being engageable with a drive tool in said first mode of operation for rotationally driving said shank and said first element in unison, said primary drive formation being engageable with the drive tool in said second mode of operation for rotationally driving said shank independently of said first element so that the first element upon rotation of said shank moves axially relative to said first element.

8. The screw of claim 4, wherein:
said first element external thread has a first element external thread outer diameter that tapers towards a leading end of said first element.

9. The screw of claim 4, wherein:
at least a leading region of said first shank thread tapers towards a trailing end of said shank and at least a leading region of said first element is configured to radially expand upon engagement with said leading region of said first shank thread.

10. The screw of claim 9, wherein:
at least a leading region of said first element is provided with one or more longitudinally extending slits to enable radial expansion of said leading region of said first element.

11. The screw of claim 5, wherein:
said screw further comprises a locking mechanism configured to lock said first element relative to said first shank portion upon installation of said compression bone screw.

12. The screw of claim 11, wherein:
said locking mechanism comprises a deformable detent secured to one of said first element and said first shank portion such that, upon installation of said compression bone screw, the other of said first element and said first shank portion engages and deforms said deformable detent to lock said first element to said first shank portion.

13. The screw of claim 11, wherein:
said locking mechanism comprises a locking device configured to engage said end face of said first shank portion and said end face of said first element to lock said first element relative to said first shank portion upon installation of said compression bone screw.

14. The screw of claim 5, wherein:
said first shank portion and said first element are each configured to releasably engage a locking member to fix said first shank portion relative to said first element for said first mode of operation.

15. The screw of claim 14, wherein:
said first shank portion is provided with a longitudinally extending shank groove and said first element is provided with a first element groove, said shank grove and said first element groove co-operating to receive said locking member in use.

16. The screw of claim 16, wherein:
first and second primary detents are provided at the end of said first and second shank portions respectively for engaging said first element and said second element respectively during said first mode of operation, thereby enabling said shank to be rotationally driven in unison with said first element and said second element in said first mode of operation, said second shank thread being opposite handed to said second element external thread.

17. A compression bone screw, comprising:
a shank having longitudinally opposing first and second shank portions, said first shank portion being externally threaded with a first shank thread, said second shank portion being externally threaded with a second shank thread, said second shank thread being opposite-handed to said first shank thread;
a first element threadingly mounted on said first shank portion by way of a first element internal thread threadingly engaging said first shank thread, said first element being externally threaded with a first element external thread; and
a second element integral with and formed by an enlargement of the second shank portion by, said second element having an external thread, said second element external thread and said first element external thread being like-handed; said shank, said first element and said second element being configured to co-operate in a first mode of operation in which said shank, said first element and said second element are rotationally driven in unison such that each advance axially in the same direction and at the same rate upon application of said rotational drive and a second mode of operation in which said shank is rotationally driven distally independent of said first element, such that said first element moves along said first shank portion towards said second element.

18. The screw of claim 22, wherein:
said first shank portion is a trailing shank portion, said second shank portion is a leading shank portion, said first element is a trailing element and said second element is a leading element.

19. A method of fixing a proximal bone fragment to a distal bone fragment, said method comprising the steps of:
a) providing a compression bone screw comprising:
(i) a shank having longitudinally opposing first and second shank portions, said first shank portion being externally threaded with a first shank thread;
(ii) a first element threadingly mounted on said first shank portion by way of a first element internal thread threadingly engaging said first shank thread, said first element being externally threaded with a first element external thread; and
(iii) a second element integrally formed with and by an enlargement of said second shank portion, said second element being externally threaded with a second element external thread, said second element external thread and said first element external thread being like-handed;
b) drilling a hole through said proximal bone fragment into said distal bone fragment;
c) rotationally driving said screw into said hole with said second element leading, rotationally driving said first element, said second element and said shank in unison until said second element is embedded within said distal bone fragment and said first element is embedded in said proximal bone fragment;
d) rotationally driving said shank independently of said first element in a direction tending to draw said first and second elements together.
